(19) United States
(12) Patent Application Publication
(10) Pub. No.: US 2025/0127405 A1
(43) Pub. Date: Apr. 24, 2025

(54) METHOD AND APPARATUS FOR MANAGING ACUTE ISCHEMIC EVENTS

(71) Applicant: DePuy Synthes Products, Inc., Raynham, MA (US)

(72) Inventors: Dillon KARG, South Lake Tahoe, CA (US); Sheila RUIZ, Mission Viejo, CA (US)

(73) Assignee: DePuy Synthes Products, Inc., Raynham, MA (US)

(21) Appl. No.: 18/382,730

(22) Filed: Oct. 23, 2023

(51) Int. Cl. A61B 5/02; A61B 5/0215; A61M 25/01

(52) U.S. Cl. CPC A61B 5/02007; A61B 5/0215; A61M 25/0147

(57) ABSTRACT

A tool for measuring a density of a clot within a vessel of a patient includes a first depth indicator wire disposed in the lumen of the flexible catheter. The second end of the first depth indicator wire extends distally beyond the second distal end of the catheter. A chamber has a piston disposed within the chamber. The proximal end of the first depth indicator wire is connected to the distal side of the piston. A spring is disposed within the chamber. A second depth indicator wire is connected to the proximal side of the piston. A proximal end of the second depth indicator wire extends proximally beyond the proximal end of the chamber and has a plurality of depth indicator markers on an exterior surface thereof.

### FIELD

The disclosure relates to acute management of an ischemic event such as a stroke. In particular, this disclosure relates to endovascular medical systems advanceable through the vasculature for managing acute ischemic events.

### BACKGROUND

The World Health Organization estimates that 15,000,000 strokes occur annually. Approximately 87% of all strokes are ischemic—where a blood clot obstructs the flow of blood to an area of the brain, starving it of oxygen and nutrients. Clots may develop and block vessels locally without being released in the form of an embolus—this mechanism is common in the formation of coronary blockages. Acute obstructions are mainly blood clots, but other sources may be fat emboli, cardiac tumors, cardiac or arterial connective tissue, misplaced devices, migrated devices, and the like. Thromboembolism occurs when part or all of a thrombus breaks away from the blood vessel wall. This clot is then carried in the direction of blood flow. Clots can include a range of morphologies and consistencies. Long strands of softer clot material may tend to lodge at bifurcations or trifurcations, resulting in multiple vessels being simultaneously occluded over significant lengths. Older clot material can also be less compressible than softer fresher clots, and under the action of blood pressure may distend the compliant vessel in which it is lodged. Clots also vary greatly in length, even if lodged in the same region of the vascular anatomy. For example, clots occluding the middle cerebral artery of an ischemic stroke patient may range from just a few millimeters to several centimeters in length.

Of the approximately 13,000,000 ischemic strokes that occur annually, one-third of patients die and another one-third are disabled. Two of the primary factors associated with mortality in these patients are the occlusion location and the time to treatment. Regarding treatment time from symptom onset, treating the occlusion as fast as practical is important to avoid complications that result in vessels deprived of flow because of the occlusion. Intravenous (IV) thrombolytics are used for patients presenting up to 4.5 hours after symptom onset. Guidelines recommend administering IV thrombolytics in the 3-4.5 hour window to those patients who meet the ECASS 3 (European Cooperative Acute Stroke Study 3) trial inclusion/exclusion criteria. As for location, large-vessel occlusions, which are present in 46% of unselected acute stroke patients presenting in academic medical centers, are associated with higher stroke severity. Additionally, not all patients may be treated with thrombolytic therapy, and so mechanical thrombectomy is a valuable alternative in patients contraindicated to t-PA (tissue plasminogen activator) or where t-PA treatment was not effective. These more proximal vessels feed a large volume of brain tissue, ergo clinicians use the presenting NIHSS (National Institute of Health Stroke Scale) score as an indicator of large-vessel occlusion.

With this, it is understood that an ischemic stroke may result if the clot lodges in the cerebral vasculature. In the United States alone, roughly 700,000 acute ischemic stroke (AIS) cases occur every year and this number is expected to increase with an ageing population. Occlusion of these large arteries in ischemic stroke is associated with significant disability and mortality. Revascularization of intracranial artery occlusions is the therapeutic goal in stroke therapy.

Endovascular mechanical revascularization (thrombectomy) is an increasingly used method or use for intracranial large vessel recanalization in acute stroke. Such devices based on stent-like technology, referred to as “stentrievers” or “stent-retrievers”, are currently displacing first generation thrombectomy devices for recanalization in acute ischemic stroke. There are significant challenges associated with designing clot removal devices that can deliver high levels of performance. There are also a number of access challenges that make it difficult to deliver devices. For example, the vasculature in the area in which the clot may be lodged is often fragile and delicate. In particular, neurovascular vessels are more fragile than similarly sized vessels in other parts of the body and are often just sparsely connected to the surrounding soft tissue bed. Excessive tensile forces applied to these vessels could result in perforations and hemorrhage. Peri-interventional subarachnoid hemorrhage (SAH) is one of the most feared complications associated with endovascular thrombectomy therapy.

There are also significant challenges associated with designing clot removal devices that can deliver high levels of performance. First, the vasculature can present a number of access challenges that make it difficult to deliver devices. In cases where access involves navigating the aortic arch (such as coronary or cerebral blockages), the configuration of the arch in some patients makes it difficult to position a guide catheter. These difficult arch configurations are classified as either type 2 or type 3 aortic arches with type 3 arches presenting the most difficulty.

The reason for poor revascularization results using current devices and approaches is multifaceted. Challenges such as the type of clot, length of the clot, vascular architecture, and patient comorbidities can play key roles. Complex vessel tortuosity in some patients, particularly in the aged can further exacerbate the difficulty of addressing these challenging occlusions. Tortuosity can make it more difficult not just to access but also to dislodge the clot, possibly due to the line of force applied to the clot by the device and the potential for vessel movement and deformation.

Regarding variation in types of clots, clots can range broadly in their composition, distribution of components (heterogeneity) and mechanical properties. The variation is due to multiple factors, including, but not limited to, the origin of the clot, clot location, age of the clot, cellular content, non-cellular content, red blood cells, platelets, and white blood cells. Non-cellular content can include factors such as fibrin, platelets, von Willebrand factor (vWF) (i.e., a blood glycoprotein involved in hemostasis), as well as collagen. Other factors can include levels of serum in the clot, calcified deposits, lipids, clot shape, clot size, heterogeneity of distribution of constituents, as well as pathogenesis.

Because of these several factors affecting clot formation a broad spectrum of clot types are observed, for example certain clots can have long strands of deformable clot material which may tend to lodge at bifurcations or trifurcations, resulting in multiple vessels being simultaneously occluded over significant lengths. More mature and organized clot material is likely to be less compressible than a softer fresher clot, and under the action of blood pressure it may distend the compliant vessel in which it is lodged. Further, as understood throughout this disclosure, clots that have a high fibrin content (e.g., higher than 40% fibrin content) can have a high coefficient of friction (e.g., be more dense or stiff and/or be connected more strongly to the vessel wall), which renders fibrin-rich clots very difficult to dislodge. Multiple passes by a device may be required to remove or even dislodge fibrin rich clots from the vasculature when compared to the relatively higher red blood cell (RBC) content clots, which can be softer and less dense or stiff. Further, the properties of the clot may be significantly changed by the action of the retrieval device interacting with the clot (e.g., after the device makes a first pass of the clot). For example, compression of a blood clot can cause dehydration of the clot and result in a dramatic increase in both clot stiffness and coefficient of friction.

While it is possible to assess many of these characteristics in vitro by examining individual clots removed during mechanical thrombectomy procedures, in vitro tests have several clear limitations. Foremost, they are time consuming and often destructive to the sample. Samples may have to undergo extensive processing to make them amenable to in vitro analysis. Significantly for some pathologies, such as acute ischemic stroke, time to treatment is a critical factor for patient outcomes and in vitro tests may not be a practical way to determine the best treatment for the patient because of the time involved with sampling and analysis.

The solution of this disclosure resolves these limitations and other issues of the art through an in vivo measurement approach to improve management of acute ischemic events.

### SUMMARY

Disclosed herein are various exemplary devices, systems, and method or uses of the present disclosure that can address the above needs. In some embodiments, a tool for measuring a density of a clot within a vessel of a patient is disclosed. The tool includes a flexible catheter having a first proximal end and a second distal end. The catheter has a lumen extending from the first proximal end to the second distal end. A first depth indicator wire has a first proximal end and a second distal end. The first depth indicator wire is disposed in the lumen of the flexible catheter. The second end of the first depth indicator wire extends distally beyond the second distal end of the catheter. A chamber has a proximal end and a distal end. The proximal end of the catheter is connected to the distal end of the chamber. The chamber has a piston disposed within the chamber. The piston has a proximal side and a distal side. The proximal end of the first depth indicator wire is connected to the distal side of the piston. A spring, disposed within the chamber, has a proximal end and a distal end. The spring is disposed between the proximal end of the chamber and the proximal side of the piston. A second depth indicator wire has a proximal end and a distal end. The distal end of the second depth indicator wire is connected to the proximal side of the piston. A proximal end of the second depth indicator wire extends proximally beyond the proximal end of the chamber. A portion of the second depth indicator wire extends proximally beyond the proximal end of the chamber has a plurality of depth indicator markers on an exterior surface thereof. The distal end of the first depth indicator wire is configured to be moved within a clot within a vessel thereby causing the first depth indicator wire to meet resistance from the clot and to move proximally relative to the chamber depending upon a density of the clot. The proximal end of the first depth indicator wire is configured to apply a force to the piston and thereby cause the piston to move distally and causing the second depth indicator wire to move proximally exposing depth indicator markers extending beyond the proximal end of the chamber.

To the accomplishment of the foregoing and related ends, certain illustrative aspects are described herein in connection with the following description and the appended drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE DRAWINGS

The disclosure will be more clearly understood from the following description of some embodiments thereof, given by way of example only with reference to the accompanying drawings in which:

FIG. 1 is a schematic view of the tool for measuring a density of a clot within a vessel of a patient in accordance with the present invention.

FIG. 2A is a schematic view of the tool having a depth indicator wire partially penetrating a relatively less dense clot.

FIG. 2B is a schematic view of the tool having a depth indicator wire partially penetrating a relatively denser clot.

FIG. 3 is a schematic view of the tool having a depth indicator wire about to penetrate a clot.

FIG. 4 is a schematic view of another embodiment of the tool for measuring a density of a clot within a vessel of a patient in accordance with the present invention.

FIG. 5 is a schematic view of another embodiment of the tool for measuring a density of a clot within a vessel of a patient in accordance with the present invention.

FIG. 6 is a flow chart illustrating a method for measuring clot density within a vessel of a patient.

### DETAILED DESCRIPTION

Some aspects of the present disclosure relate to method or uses and systems for analyzing and/or classifying acute ischemic events, in vivo and/or in vitro, as well as individualizing a treatment protocol for the particular acute ischemic event. Although example embodiments of the present disclosure are explained in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

As discussed herein, the terms “distal” or “proximal” are used in the following description with respect to a position or direction relative to the treating physician or medical interventionalist. “Distal” or “distally” are a position distant from or in a direction away from the physician or interventionalist. “Proximal” or “proximally” or “proximate” are a position near or in a direction toward the physician or medical interventionist. The terms “occlusion”, “clot”, “thrombus” or “blockage” are used interchangeably.

As discussed herein, a “subject” or “patient” may be any applicable human, animal, or other organism, living or dead, or other biological or molecular structure or chemical environment, and may relate to particular components of the subject, for instance specific tissues or fluids of a subject (e.g., human tissue in a particular area of the body of a living subject), which may be in a particular location of the subject, referred to herein as an “area of interest” or a “region of interest.”

As discussed herein, a “sensor” may be any device or element of a device that can detect or measuring or reading or storing or otherwise communicating a physical property of the clot or vasculature or other feature of a subject of this disclosure.

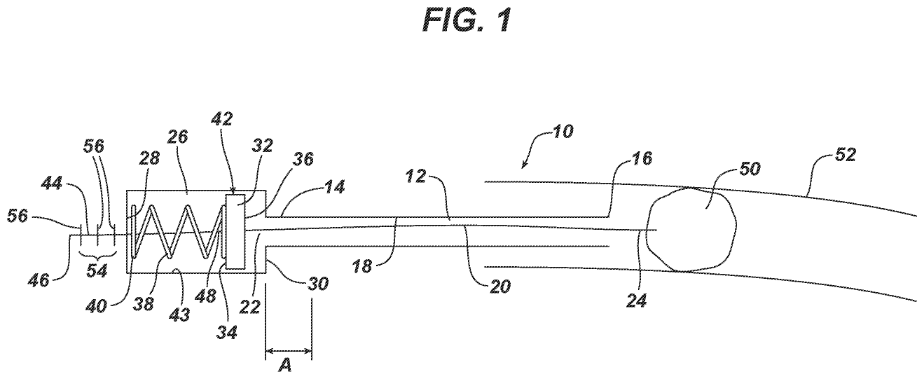

Referring now to FIG. 1, a tool 10 for measuring a density of a clot 50 within a vessel 52 of a patient is disclosed. The tool includes a flexible catheter 12 having a first proximal end 14 and a second distal end 16. Catheter 12 has a lumen 18 extending from the first proximal end 14 to the second distal end 16. A first depth indicator wire 20 has a first proximal end 22 and a second distal end 24. The first depth indicator wire 20 is disposed in the lumen 18 of the flexible catheter 12. The second end 24 of the first depth indicator wire 20 extends distally beyond the second distal end 16 of catheter 12.

A chamber 26 has a proximal end 28 and a distal end 30. The proximal end 14 of catheter 12 is connected to the distal end 30 of chamber 26. Chamber 26 has a piston 32 disposed within the chamber. The piston 32 has a proximal side 34 and a distal side 36. The proximal end 22 of the first depth indicator wire 20 is connected to the distal side 36 of piston 32. A spring 38, disposed within chamber 26, has a proximal end 40 and a distal end 42. Spring 38 is disposed between the proximal end 28 of chamber 26 and the proximal side 34 of piston 32.

A second depth indicator wire 44 has a proximal end 46 and a distal end 48. The distal end 48 of the second depth indicator wire 44 is connected to the proximal side 34 of piston 32. A proximal end 46 of the second depth indicator wire 44 extends proximally beyond the proximal end 28 of chamber 26. A portion 54 of the second depth indicator wire 44 extends proximally beyond the proximal end 28 of chamber 26 has a plurality of depth indicator markers 56 on an exterior surface thereof. Spring 38 is preferably a coil spring. A portion of the second depth indicator wire 44 is disposed within chamber 26 and is disposed within a lumen of coil spring 38. The plurality of depth indicator markers 56 are disposed on the proximal end 46 of the second depth indicator wire 44.

The distal end 24 of the first depth indicator wire 20 is configured to be moved within a clot 50 within a vessel 52 thereby causing the first depth indicator wire 20 to meet resistance from clot 50 and to move distally depending upon a density of clot 50. The proximal end 22 of the first depth indicator wire 20 is configured to apply a force to piston 32 and thereby cause the piston to move distally and causing the second depth indicator wire 44 to move proximally exposing depth indicator markers 56 extending beyond the proximal end 28 of chamber 26.

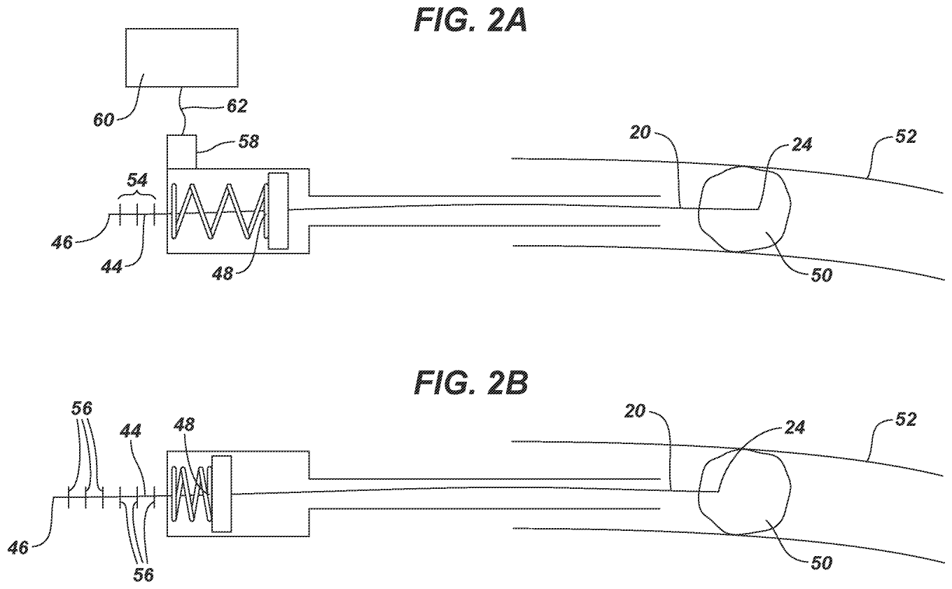

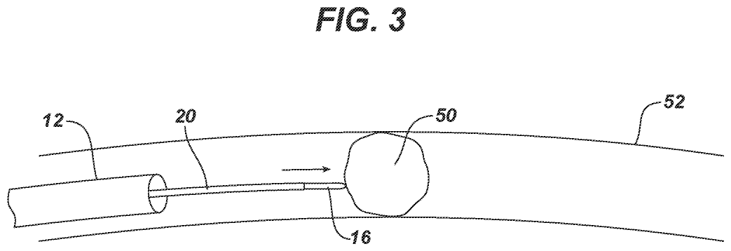

Referring now to FIGS. 1, 2A, 2B and 3, tool 10 is shown in FIG. 3 with first depth indicator wire 20 is disposed adjacent to clot 50 but has not yet penetrated the clot. In this position, the user can move chamber 26 distally a predetermined distance A as shown in FIG. 1. Depending upon the density of the clot, the distal end 24 of the first depth indicator wire 20 will penetrate clot 50 a different distance as illustrated in FIGS. 2A and 2B. Clot 50 shown in FIG. 2A is less dense and thus provides less resistance to wire 20. Thus spring 38 is compressed to a lesser extent and less markers 56 are exposed beyond the proximal end 28 of chamber 26. Clot 50 shown in FIG. 2B is relatively denser than the clot of FIG. 2A and thus provides more resistance to wire 20. Thus spring 38 is compressed to a greater extent and more markers 56 are exposed beyond the proximal end 28 of chamber 26. The system is calibrated so the user will know the relative density of the clot. In addition, a sensor 58 may be used to determine the number of depth indicator markers 56 extending beyond the proximal end 28 of chamber 26 and to send a signal to the display 60 indicative of the number of depth indicator markers extending beyond the proximal end 28 of chamber 26. Display 60 includes a screen configured to receive input from sensor 58 and provides a visual output to the user of the number of depth indicator markers 56 extending beyond the proximal end 28 of chamber 26. Display 60 is connected to sensor 58 either by a wired connection 62 or by a wireless connection as is known in the art.

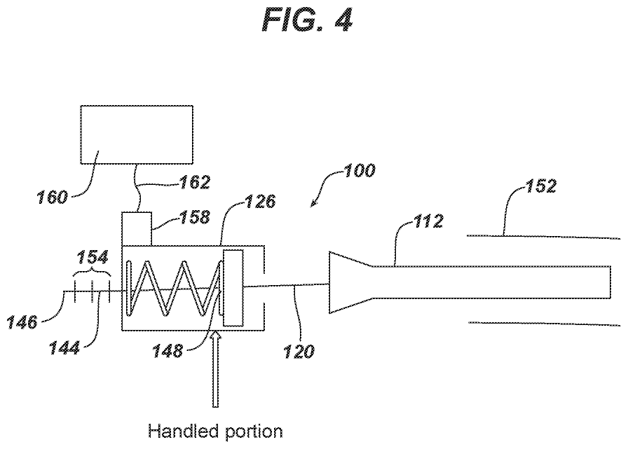

Referring now to FIG. 4, another embodiment of the tool 100 for measuring a density of a clot within a vessel of a patient in accordance with the present invention is illustrated. For the sake of brevity, only those portions of this embodiment that differ from the FIGS. 1-3 embodiment will be described. In this embodiment, tool 100 is not directly connected to the flexible catheter 112. An off the shelf catheter 112 may be used to access the site within the vasculature as is known in the art. Once catheter 112 is in place, the user may insert first depth indicator wire 120 within and through the lumen of catheter 112 in the same manner as the embodiment described above and illustrated in FIGS. 1-3.

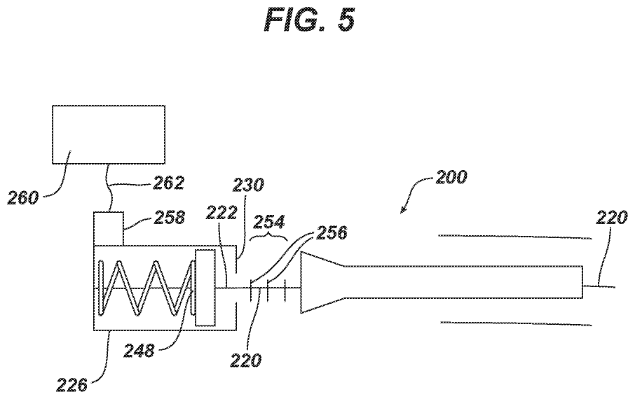

Referring now to FIG. 5, another embodiment of the tool 200 for measuring a density of a clot within a vessel of a patient in accordance with the present invention is illustrated. For the sake of brevity, only those portions of this embodiment that differ from the FIGS. 1-3 embodiment will be described. In this embodiment, tool 200 is also not directly connected to the flexible catheter 212. An off the shelf catheter 212 may be used to access the site within the vasculature as is known in the art. Once catheter 212 is in place, the user may insert first depth indicator wire 220 within and through the lumen of catheter 212 in the same manner as the embodiment described above and illustrated in FIGS. 1-3. However, in this embodiment the markers 256 are on the proximal end 222 of the first depth indicator wire 220. Thus, for a less dense clot 250, more markers 256 will be exposed beyond the distal end 230 of chamber 226. Likewise, less markers will extend beyond the distal end 230 of chamber 226 for a denser clot 250.

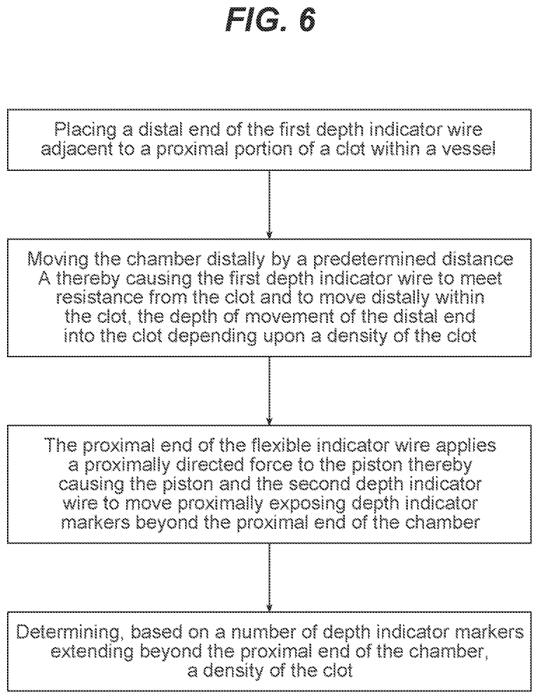

A method for measuring the clot density of a clot within a vessel of a patient includes providing a tool 10 as described above. Referring now to FIG. 6, the method includes the step of placing a distal end of the first depth indicator wire adjacent to a proximal portion of a clot within a vessel. The chamber 26 is moved distally a predetermined distance A as shown in FIG. 1. Alternatively, a predetermined force in the distal direction can be applied to chamber 26. Moving chamber 26 by distance A causes the first depth indicator wire 20 to move in the distal direction thereby causing the distal end 24 of the first depth indicator wire to meet resistance from the clot 50 and to move distally within the clot. The depth of movement of the distal end 24 into clot 50 depends upon a density of the clot. The proximal end 22 of the flexible indicator wire 20 is connected to piston 32. Wire 20 will apply a proximally directed force to the piston 32 within chamber 26 thereby causing the piston and the second depth indicator wire to move proximally relative to chamber 26 thereby exposing depth indicator markers 56 beyond the proximal end 28 of chamber 26. Of course, piston 32 will move in the distal direction, but not as far as distance A depending upon the density of clot 50. Thus, with respect to chamber 26, piston 32 appears to be moving proximally. A density of clot 50 can be determined, based on a number of depth indicator markers 56 extending beyond the proximal end of chamber 26.

A technique for removing the clot from the vessel can be determined based upon the measured density of the clot. A stent retriever may be used to remove the clot. Alternatively, aspiration may be used to remove the clot, or a combination of aspiration and a stent retriever may be used to remove the clot.

Upon determining the relative density of the clot 50, 150, 250 the physician or one of her assistants may then choose the proper course of treatment for the clot, ranging from, for example, using aspiration, a stent retriever, a pinch retriever, a drug to dissolve the clot, or a combination thereof. The stent retriever may be, for example, the EMBOTRAP III™ revascularization device that is commercially available from Cerenovus.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method or use step is present in the composition or article or method or use, but does not exclude the presence of other compounds, materials, particles, method or use steps, even if the other such compounds, material, particles, method or use steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method or use does not preclude the presence of additional method or use steps or intervening method or use steps between those steps expressly identified. Steps of a method or use may be performed in a different order than those described herein without departing from the scope of the present disclosure. Similarly, it is also to be understood that the mention of one or more components in a device or system does not preclude the presence of additional components or intervening components between those components expressly identified.

The descriptions contained herein are examples illustrating the solution and are not intended to limit the scope. As described herein, the solution contemplates many variations and modifications of a system, device, and/or method or use that can be used to analyze one or more clots and individualize treatment based on the analysis. Variations can include but are not limited to alternative geometries of elements and components described herein, utilizing any of numerous materials for each component or element (e.g. radiopaque materials, memory shape metals, etc.), utilizing additional components, utilizing additional components to perform functions described herein, or utilizing additional components to perform functions not described herein, for example. These modifications would be apparent to those having ordinary skill in the art to which this invention relates and are intended to be within the scope of the claims which follow.

The specific configurations, choice of materials and the size and shape of various elements can be varied according to particular design specifications or constraints requiring a system or method or use constructed according to the principles of the disclosed technology. Such changes are intended to be embraced within the scope of the disclosed technology. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein.

The following clauses list non-limiting embodiments of the disclosure:
1. A tool for measuring a density of a clot within a vessel of a patient, the tool comprising:
   a flexible catheter having a first proximal end and a second distal end, the catheter having a lumen extending from the first proximal end to the second distal end;
   a first depth indicator wire having a first proximal end and a second distal end, the first depth indicator wire being disposed in the lumen of the flexible catheter, the second end of the first depth indicator wire extending distally beyond the second distal end of the catheter;
   a chamber having a proximal end and a distal end; the proximal end of the catheter being connected to the distal end of the chamber, the chamber having a piston disposed within the chamber, the piston having a proximal side and a distal side; the proximal end of the first depth indicator wire being connected to the distal side of the piston;
   a spring being disposed within the chamber, the spring having a proximal end and a distal end, the spring being disposed between the proximal end of the chamber and the proximal side of the piston;
   a second depth indicator wire having a proximal end and a distal end, the distal end of the second depth indicator wire being connected to the proximal side of the piston, a proximal end of the second depth indicator wire extending proximally beyond the proximal end of the chamber, a portion of the second depth indicator wire extending proximally beyond the proximal end of the chamber having a plurality of depth indicator markers on an exterior surface thereof;
   wherein the distal end of the first depth indicator wire is configured to be moved within a clot within a vessel thereby causing the first depth indicator wire to meet resistance from the clot and to move proximally depending upon a density of the clot, the proximal end of the first depth indicator wire configured to apply a force to the piston and thereby cause the piston to move distally and causing the second depth indicator wire to move proximally exposing depth indicator markers beyond the proximal end of the chamber.
2. The tool for measuring clot density according to clause 1, wherein the plurality of depth indicator markers are disposed on the proximal end of the second depth indicator wire.
3. The tool for measuring clot density according to clauses 1 or 2, further comprising:
   a sensor and a display, the sensor being configured to determine the number of depth indicator markers extending beyond the proximal end of the chamber and to send a signal to the display indicative of the number of depth indicator markers extending beyond the proximal end of the chamber, the display including a screen configured to receive input from the sensor and to provide a visual output to the user of the number of depth indicator markers extending beyond the proximal end of the chamber.
4. The tool for measuring clot density according to any of clauses 1-3, wherein the spring is a coil spring, wherein a portion of the second depth indicator wire disposed within the chamber is disposed within the coil spring.
5. A tool for measuring a density of a clot within a vessel of a patient, the tool comprising:
   a flexible catheter having a first proximal end and a second distal end, the catheter having a lumen extending from the first proximal end to the second distal end;
   a first depth indicator wire having a first proximal end and a second distal end, the wire being mounted in the lumen of the flexible catheter, the second distal end of the wire extending distally beyond the second distal end of the catheter;
   a chamber having a proximal end and a distal end; the proximal end of the catheter connected to the distal end of the chamber; the chamber having a piston disposed within the chamber; the piston having a proximal side, a distal side and an outer circumferential surface in scaling contact with an inner wall surface of the chamber; the proximal end of the first depth indicator wire connected to the distal side of the piston;
   a compressible gas being disposed within the chamber between the proximal end of the chamber and the proximal side of the piston;
   a second depth indicator wire having a proximal end and a distal end, the distal end of the second depth indicator wire being connected to the proximal side of the piston, a proximal end of the second depth indicator wire extending proximally beyond the proximal end of the chamber, a portion of the second depth indicator wire extending proximally beyond the proximal end of the chamber having a plurality of depth indicator markers disposed on an exterior surface thereof, an outer circumferential surface of the second depth indicator wire in sealing contact with an opening in a proximal end wall of the chamber;
   wherein the distal end of the first depth indicator wire is configured to be placed within a clot within a vessel by a predetermined distance thereby causing the flexible indicator wire to meet resistance from the clot and to move proximally depending upon a density of the clot, the proximal end of the first flexible indicator wire being configured to apply a force to the piston and thereby cause the piston to move proximally, thereby causing the second depth indicator wire to move proximally exposing depth indicator markers beyond the proximal end of the chamber.
6. The tool for measuring clot density according to clause 5, wherein the plurality of depth indicator markers are disposed on the proximal end of the second depth indicator wire.
7. The tool for measuring clot density according to clauses 5 or 6, further comprising:
   a sensor and a display, the sensor being configured to determine the number of depth indicator markers extending beyond the proximal end of the chamber and to send a signal to the display indicative of the number of depth indicator markers extending beyond the proximal end of the chamber, the display including a screen configured to receive input from the sensor and to provide a visual output to the user of the number of depth indicator markers extending beyond the proximal end of the chamber.
8. A tool for measuring a density of a clot within a vessel of a patient, the tool comprising:
   a guide wire having a first proximal end and a second distal end;
   a chamber having a proximal end and a distal end; the proximal end of the guide wire slidingly connected to the distal end of the chamber; the chamber having a piston disposed within the chamber; the piston having a proximal side and a distal side; the proximal end of the guide wire connected to the distal side of the piston;
   a spring being disposed within the chamber, the spring having a proximal end and a distal end, the spring being disposed between the proximal end of the chamber and the proximal side of the piston;
   a depth indicator wire having a proximal end and a distal end, the distal end of the depth indicator wire being connected to the proximal side of the piston, a proximal end of the depth indicator wire extending proximally beyond the proximal end of the chamber, a portion of the depth indicator wire extending proximally beyond the proximal end of the chamber having a plurality of depth indicator markers on an exterior surface thereof;
   wherein the distal end of the guide wire is configured to be moved within a clot within a vessel thereby causing the guide wire to meet resistance from the clot and to move distally depending upon a density of the clot, the proximal end of the guidewire configure to apply a force to the piston and thereby cause the piston to move proximally and causing the depth indicator wire to move proximally exposing depth indicator markers beyond the proximal end of the chamber.
9. The tool for measuring clot density according to clause 8, wherein the plurality of depth indicator markers are disposed on the proximal end of the depth indicator wire.
10. The tool for measuring clot density according to clause 8 or 9, further comprising:
a sensor and a display, the sensor being configured to determine the number of depth indicator markers extending beyond the proximal end of the chamber and to send a signal to the display indicative of the number of depth indicator markers extending beyond the proximal end of the chamber, the display including a screen configured to receive input from the sensor and to provide a visual output to the user of the number of depth indicator markers extending beyond the proximal end of the chamber.
11. The tool for measuring clot density according to any of clauses 8-10, wherein the spring is a coil spring, wherein a portion of the second depth indicator wire disposed within the chamber is disposed within the coil spring.
12. A tool for measuring a density of a clot within a vessel of a patient, the tool comprising:
a guide wire having a first proximal end and a second distal end;
a chamber having a proximal end and a distal end; the proximal end of the guide wire slidingly connected to the distal end of the chamber; the chamber having a piston disposed within the chamber; the piston having a proximal side and a distal side; the proximal end of the guide wire connected to the distal side of the piston;
a spring being disposed within the chamber, the spring having a proximal end and a distal end, the spring being disposed between the proximal end of the chamber and the proximal side of the piston;
wherein, the proximal end of the guide wire proximal end of the depth indicator wire extending distally beyond the distal end of the chamber, a portion of the depth indicator wire extending distally beyond the distal end of the chamber having a plurality of depth indicator markers on an exterior surface thereof; wherein the distal end of the guide wire is configured to be moved within a clot within a vessel thereby causing the guide wire to meet resistance from the clot and to move distally depending upon a density of the clot, the proximal end of the guidewire configure to apply a force to the piston and thereby cause the piston to move proximally and causing the guide wire to move proximally exposing depth indicator markers beyond the distal end of the chamber.
13. The tool for measuring clot density according to clause 12, further comprising:
a sensor and a display, the sensor being configured to determine the number of depth indicator markers extending beyond the distal end of the chamber and to send a signal to the display indicative of the number of depth indicator markers extending beyond the distal end of the chamber, the display including a screen configured to receive input from the sensor and to provide a visual output to the user of the number of depth indicator markers extending beyond the distal end of the chamber.
14. The tool for measuring clot density according to clause 12 or 13, wherein the spring is a coil spring.
15. A method for measuring clot density within a vessel of a patient, the method comprises the steps of:
providing a tool comprising:
a flexible catheter having a first proximal end and a second distal end, the catheter having a lumen extending from the first proximal end to the second distal end;
a first depth indicator wire having a first proximal end and a second distal end, the first depth indicator wire being disposed in the lumen of the flexible catheter, the second end of the first depth indicator wire extending distally beyond the second distal end of the catheter;
a chamber having a proximal end and a distal end; the proximal end of the catheter connected to the distal end of the chamber; the chamber having a piston disposed within the chamber; the piston having a proximal side and a distal side; the proximal end of the first depth indicator wire connected to the distal side of the piston;
a spring being disposed within the chamber, the spring having a proximal end and a distal end, the spring being disposed between the proximal end of the chamber and the proximal side of the piston;
a second depth indicator wire having a proximal end and a distal end, the distal end of the second depth indicator wire being connected to the proximal side of the piston, a proximal end of the second depth indicator wire extending proximally beyond the proximal end of the chamber, a portion of the second depth indicator wire extending proximally beyond the proximal end of the chamber having a plurality of depth indicator markers on an exterior surface thereof;
wherein the distal end of the first depth indicator wire is configured to be moved within a clot within a vessel thereby causing the first depth indicator wire to meet resistance from the clot and to move proximally depending upon a density of the clot, the proximal end of the first depth indicator wire configure to apply a force to the piston and thereby cause the piston to move proximally and causing the second depth indicator wire to move proximally exposing depth indicator markers beyond the proximal end of the chamber;
placing a distal end of the first depth indicator wire adjacent to a proximal portion of a clot within a vessel;
moving the chamber distally a predetermined distance A or alternatively applying a predetermined force to the first depth indicator wire in the distal direction thereby causing the first depth indicator wire to meet resistance from the clot and to move proximally within the clot, the depth of movement of the distal end into the clot depending upon a density of the clot, wherein the proximal end of the flexible indicator wire applies a proximally directed force to the piston thereby causing the piston and the second depth indicator wire to move proximally exposing depth indicator markers beyond the proximal end of the chamber; and
determining, based on a number of depth indicator markers extending beyond the proximal end of the chamber, a density of the clot.
16. The method for measuring clot density within a vessel of a patient according to clause 15, further comprising the step of:
determining a technique for removing the clot from the vessel based upon the measured density of the clot.
17. The method for measuring clot density within a vessel of a patient according to clause 16, further comprising the step of:
using a stent retriever to remove the clot.
18. The method for measuring clot density within a vessel of a patient according to clause 16, further comprising the step of:
aspirating the catheter to remove the clot.
19. The method for measuring clot density within a vessel of a patient according to clause 16, further comprising the step of:
aspirating the catheter and using a stent retriever to remove the clot.

What is claimed is:

1. A tool for measuring a density of a clot within a vessel of a patient, the tool comprising:
a flexible catheter having a first proximal end and a second distal end, the catheter having a lumen extending from the first proximal end to the second distal end;
a first depth indicator wire having a first proximal end and a second distal end, the first depth indicator wire being disposed in the lumen of the flexible catheter, the second end of the first depth indicator wire extending distally beyond the second distal end of the catheter;
a chamber having a proximal end and a distal end; the proximal end of the catheter being connected to the distal end of the chamber, the chamber having a piston disposed within the chamber, the piston having a proximal side and a distal side; the proximal end of the first depth indicator wire being connected to the distal side of the piston;
a spring being disposed within the chamber, the spring having a proximal end and a distal end, the spring being disposed between the proximal end of the chamber and the proximal side of the piston;
a second depth indicator wire having a proximal end and a distal end, the distal end of the second depth indicator wire being connected to the proximal side of the piston, a proximal end of the second depth indicator wire extending proximally beyond the proximal end of the chamber, a portion of the second depth indicator wire extending proximally beyond the proximal end of the chamber having a plurality of depth indicator markers on an exterior surface thereof;
wherein the distal end of the first depth indicator wire is configured to be moved within a clot within a vessel thereby causing the first depth indicator wire to meet resistance from the clot and to move proximally depending upon a density of the clot, the proximal end of the first depth indicator wire configured to apply a force to the piston and thereby cause the piston to move distally and causing the second depth indicator wire to move proximally exposing depth indicator markers beyond the proximal end of the chamber.

2. The tool for measuring clot density according to claim 1, wherein the plurality of depth indicator markers are disposed on the proximal end of the second depth indicator wire.

3. The tool for measuring clot density according to claim 1, further comprising:
a sensor and a display, the sensor being configured to determine the number of depth indicator markers extending beyond the proximal end of the chamber and to send a signal to the display indicative of the number of depth indicator markers extending beyond the proximal end of the chamber, the display including a screen configured to receive input from the sensor and to provide a visual output to the user of the number of depth indicator markers extending beyond the proximal end of the chamber.

4. The tool for measuring clot density according to claim 1, wherein the spring is a coil spring, wherein a portion of the second depth indicator wire disposed within the chamber is disposed within the coil spring.

5. A tool for measuring a density of a clot within a vessel of a patient, the tool comprising:
a flexible catheter having a first proximal end and a second distal end, the catheter having a lumen extending from the first proximal end to the second distal end;
a first depth indicator wire having a first proximal end and a second distal end, the wire being mounted in the lumen of the flexible catheter, the second distal end of the wire extending distally beyond the second distal end of the catheter;
a chamber having a proximal end and a distal end; the proximal end of the catheter connected to the distal end of the chamber; the chamber having a piston disposed within the chamber; the piston having a proximal side, a distal side and an outer circumferential surface in sealing contact with an inner wall surface of the chamber; the proximal end of the first depth indicator wire connected to the distal side of the piston;
a compressible gas being disposed within the chamber between the proximal end of the chamber and the proximal side of the piston;
a second depth indicator wire having a proximal end and a distal end, the distal end of the second depth indicator wire being connected to the proximal side of the piston, a proximal end of the second depth indicator wire extending proximally beyond the proximal end of the chamber, a portion of the second depth indicator wire extending proximally beyond the proximal end of the chamber having a plurality of depth indicator markers disposed on an exterior surface thereof, an outer circumferential surface of the second depth indicator wire in sealing contact with an opening in a proximal end wall of the chamber;
wherein the distal end of the first depth indicator wire is configured to be placed within a clot within a vessel by a predetermined distance thereby causing the flexible indicator wire to meet resistance from the clot and to move proximally depending upon a density of the clot, the proximal end of the first flexible indicator wire being configured to apply a force to the piston and thereby cause the piston to move proximally, thereby causing the second depth indicator wire to move proximally exposing depth indicator markers beyond the proximal end of the chamber.

6. The tool for measuring clot density according to claim 5, wherein the plurality of depth indicator markers are disposed on the proximal end of the second depth indicator wire.

7. The tool for measuring clot density according to claim 5, further comprising:
a sensor and a display, the sensor being configured to determine the number of depth indicator markers extending beyond the proximal end of the chamber and to send a signal to the display indicative of the number of depth indicator markers extending beyond the proximal end of the chamber, the display including a screen configured to receive input from the sensor and to provide a visual output to the user of the number of depth indicator markers extending beyond the proximal end of the chamber.

8. A tool for measuring a density of a clot within a vessel of a patient, the tool comprising:
a guide wire having a first proximal end and a second distal end;
a chamber having a proximal end and a distal end; the proximal end of the guide wire slidingly connected to the distal end of the chamber; the chamber having a piston disposed within the chamber; the piston having a proximal side and a distal side; the proximal end of the guide wire connected to the distal side of the piston;
a spring being disposed within the chamber, the spring having a proximal end and a distal end, the spring being disposed between the proximal end of the chamber and the proximal side of the piston;
a depth indicator wire having a proximal end and a distal end, the distal end of the depth indicator wire being connected to the proximal side of the piston, a proximal end of the depth indicator wire extending proximally beyond the proximal end of the chamber, a portion of the depth indicator wire extending proximally beyond the proximal end of the chamber having a plurality of depth indicator markers on an exterior surface thereof;
wherein the distal end of the guide wire is configured to be moved within a clot within a vessel thereby causing the guide wire to meet resistance from the clot and to move distally depending upon a density of the clot, the proximal end of the guidewire configure to apply a force to the piston and thereby cause the piston to move proximally and causing the depth indicator wire to move proximally exposing depth indicator markers beyond the proximal end of the chamber.

9. The tool for measuring clot density according to claim 8, wherein the plurality of depth indicator markers are disposed on the proximal end of the depth indicator wire.

10. The tool for measuring clot density according to claim 8, further comprising:
a sensor and a display, the sensor being configured to determine the number of depth indicator markers extending beyond the proximal end of the chamber and to send a signal to the display indicative of the number of depth indicator markers extending beyond the proximal end of the chamber, the display including a screen configured to receive input from the sensor and to provide a visual output to the user of the number of depth indicator markers extending beyond the proximal end of the chamber.

11. The tool or measuring clot density according to claim 8, wherein the spring is a coil spring, wherein a portion of the second depth indicator wire disposed within the chamber is disposed within the coil spring.

12. A tool for measuring a density of a clot within a vessel of a patient, the tool comprising:
a guide wire having a first proximal end and a second distal end;
a chamber having a proximal end and a distal end; the proximal end of the guide wire slidingly connected to the distal end of the chamber; the chamber having a piston disposed within the chamber; the piston having a proximal side and a distal side; the proximal end of the guide wire connected to the distal side of the piston;
a spring being disposed within the chamber, the spring having a proximal end and a distal end, the spring being disposed between the proximal end of the chamber and the proximal side of the piston;
wherein, the proximal end of the guide wire proximal end of the depth indicator wire extending distally beyond the distal end of the chamber, a portion of the depth indicator wire extending distally beyond the distal end of the chamber having a plurality of depth indicator markers on an exterior surface thereof;
wherein the distal end of the guide wire is configured to be moved within a clot within a vessel thereby causing the guide wire to meet resistance from the clot and to move distally depending upon a density of the clot, the proximal end of the guidewire configure to apply a force to the piston and thereby cause the piston to move proximally and causing the guide wire to move proximally exposing depth indicator markers beyond the distal end of the chamber.

13. The tool for measuring clot density according to claim 12, further comprising:
a sensor and a display, the sensor being configured to determine the number of depth indicator markers extending beyond the distal end of the chamber and to send a signal to the display indicative of the number of depth indicator markers extending beyond the distal end of the chamber, the display including a screen configured to receive input from the sensor and to provide a visual output to the user of the number of depth indicator markers extending beyond the distal end of the chamber.

14. The tool for measuring clot density according to claim 12, wherein the spring is a coil spring.
